(19) United States
(12) Patent Application Publication
(10) Pub. No.: US 2002/0177848 A1
(43) Pub. Date: Nov. 28, 2002

(54) Electrosurgical working end for sealing tissue

(76) Inventors: Csaba Truckai, Saratoga, CA (US); Scott Rader, Menlo Park, CA (US); John H. Shadduck, Tiburon, CA (US)

Correspondence Address: Csaba Truckai, 19566 Arden Court, Saratoga, CA, 95070, US

(21) Appl. No.: 09/865,850

(22) Filed: May 24, 2001

(51) Int. Cl. A61B 18/18

(52) U.S. Cl. 606/50

(57) ABSTRACT

An electrosurgical working end and method for transecting an anatomic structure along a targeted line and for creating a thermal welds along either of both transected tissue margins, for example, for use in a partial lung resection procedure. The working end provides elongate guide members that can be positioned on opposing sides of the targeted anatomic structure. The working end carries a slidable extension member with interior channels that receive the guide members. The extension member can be moved from a retracted position to an extended position by advancing over the guide members. As the extension member advances over the guide members (i) the guides compress the tissue just ahead of the advancing extension member to allow the laterally-outward portions of the extension member to ramp over the tissue, (ii) while contemporaneously a cutting element at the distal end of the extension member transects the tissue. By this means, the transected tissue margins are captured under very high compression by working end components on either side of the tissue margin. The working end carries a bi-polar electrode arrangement that engages the just-transected medial tissue layers as well as surface layers to provides Rf current flow for tissue welding purposes that is described as a medial-to-surface bi-polar approach. The system also provides at least one indicator means for indicating to the surgeon when the extension member has been fully actuated, since endoscopic viewing of the very elongate guide members may not be possible.

### CROSS-REFERENCE TO RELATED APPLICATIONS

This application is a continuation-in-part of co-pending U.S. patent application Ser. No. ______ filed Feb. 24, 2001 (Docket No. SRX-006) titled Electrosurgical Working End for Transecting and Sealing Tissue. This application also is related to the following co-pending U.S. patent application Ser. No. ______ filed Oct. 23, 2000 (Docket No. SRX-001) titled Electrosurgical Systems and Techniques for Sealing Tissue; Ser. No. ______ filed Dec. 14, 2000 (Docket No. SRX-002) titled Electrosurgical Jaws for Controlled Application of Clamping Pressure. All the above-listed patent applications are incorporated herein by this reference.

### BACKGROUND OF THE INVENTION

1. Field of the Invention

This invention relates to medical devices and more particularly relates to the working end of an electrosurgical instrument that is adapted for sealing or welding tissue that is engaged between paired members wherein the working end provides highly elongate guide or jaws members for guiding a tissue-compressing member over tissue to apply high compressive forces to engaged tissue. The invention provides indicator means that can indicate to the surgeon the complete closure of the tissue-engaging system, which can be difficult to ascertain by visual observation of very elongate guides or jaws.

2. Description of the Related Art

In various open and laparoscopic surgeries, it is necessary to weld or seal the margins of transected tissue volumes, for example, in a lung resection. In some procedures, stapling instruments are used to apply a series of mechanically deformable staples to seal the transected edge a tissue volume. Such mechanical devices may create an imperfect seal that leaks which can result in later complications.

Various radiofrequency (Rf) surgical instruments have been developed for sealing the edges of transected tissues. For example, FIG. 1A shows a sectional view of the paired electrode-jaws 2a and 2b of a typical prior art bi-polar Rf grasper that engages two tissue layers. In a typical bi-polar jaw arrangement, each jaw face comprises an electrode and Rf current flows across the tissue between the first and second polarities in the opposing jaws that engage opposing exterior surfaces of the tissue. Each jaw in FIG. 1A has a central slot adapted to receive a reciprocating blade member as is known in the art for transecting the captured vessel after it is sealed. FIG. 1A depicts bi-polar Rf current flow at any point in which the Rf flow will be in flux along random paths along lines of least resistance. The Rf flow is likely to extend well into collateral tissues.

While bi-polar graspers as in FIG. 1A can adequately seal or weld tissue volumes that have a small cross-section, such bi-polar instruments are often ineffective in sealing or welding many types of anatomic structures, for example, anatomic structures having walls with irregular or thick fibrous content; bundles of disparate anatomic structures, substantially thick anatomic and structures, and large diameter blood vessels having walls with thick fascia layers.

As depicted in FIG. 1A, a prior art grasper-type instrument is depicted with jaw-electrodes engaging opposing sides of a tissue volume with substantially thick, dense and non-uniform fascia layers underlying its exterior surface, for example, a large diameter blood vessel. As depicted in FIG. 1A, the fascia layers f prevent a uniform flow of current from the first exterior tissue surface s to the second exterior tissue surface s that are in contact with electrodes 2a and 2b. The lack of uniform bi-polar current across the fascia layers f causes non-uniform thermal effects that typically result in localized tissue desiccation and charring indicated at c. Such tissue charring can elevate impedance levels in the captured tissue so that current flow across the tissue is terminated altogether. FIG. 1B depicts an exemplary result of attempting to create a weld across tissue with thick fascia layers f with a prior art bi-polar instrument. FIGS. 1A-1B show localized surface charring c and non-uniform weld regions w in the medial layers m of vessel. Further, FIG. 1B depicts a common undesirable characteristic of prior art welding wherein thermal effects propagate laterally from the targeted tissue causing unwanted collateral (thermal) damage indicated at d.

What is needed is an instrument working end that can utilize Rf energy in new delivery modalities: (i) to weld or seal tissue volumes that are not uniform in hydration, density and collagenous content; (ii) to weld a targeted tissue region while substantially preventing collateral thermal damage in regions lateral to the targeted tissue; (iii) to weld a transected margin of a bundle of disparate anatomic structures; and (iv) to weld a transected margin of a substantially thick anatomic structure.

### SUMMARY OF THE INVENTION

The object of the present invention is to provide an instrument and working end that is capable of transecting tissue and highly compressing tissue to allow for controlled Rf energy delivery to the transected tissue margins. The objective of the invention is to effectively weld tissues that have thick fascia layers or other layers with non-uniform fibrous content. Such tissues are difficult to seal since the fascia layers can prevent uniform current flow and uniform ohmic heating of the tissue.

As background, the biological mechanisms underlying tissue fusion by means of thermal effects are not fully understood. In general, the delivery of Rf energy to a captured tissue volume elevates the tissue temperature and thereby at least partially denatures proteins in the tissue. One objective is to denature such proteins, including collagen, into a proteinaceous amalgam that intermixes and fuses together as the proteins renature. As the treated region heals over time, the so-called weld is reabsorbed by the body's wound healing process.

In order to create an effective weld in a tissue volume dominated by the fascia layers, it has been found that several factors are critical. It is necessary to create a substantially even temperature distribution across the targeted tissue volume to create a uniform weld or seal. Fibrous tissue layers (i e., fascia) conduct Rf current differently than adjacent less-fibrous layers, and it is believed that differences in extracellular fluid content in such adjacent tissues also contribute greatly to the differences in ohmic heating. It has been found that by applying very high compressive forces to fascia layers and underlying non-fibrous layers, the extracellular fluids migrate from the site to collateral regions. Thus, the compressive forces can make resistance more uniform regionally within the engaged tissue. Further, it has been found that that another critical factor in creating an effective weld across fibrous (fascia) layers is the delivery of bi-polar Rf energy from electrode surfaces engaging medial layers and surface (fascia) layers. In other words, effective current flow through the fascia layers is best accomplished by engaging electrodes on opposing sides of such fascia layers. Prior art jaw structures that only deliver bi-polar Rf energy from outside the surface or fascial layers cannot cause effective regional heating inward of such fascial layers. For this reason, the novel technique causes Rf current flow to-and-from the medial (or just-transected) non-fascia layers of tissue at the interior of the structure, rather than to-and-from exterior surfaces only as in the prior art. This method is termed herein a medial-to-surface bi-polar delivery approach or a subfascia-to-fascia bi-polar approach.

The apparatus of the invention provides means for creating high compression forces over a very elongate working end that engages the targeted tissue. This is accomplished by providing a novel slidable extension member that defines channels therein that engage the entire length of elongate guide members that guide the extension member over the tissue. The extension member of the invention thus is adapted to provide multiple novel functionality: (i) to transect the tissue, and (ii) contemporaneously to engage the transected tissue margins under high compression within the components of the working end. Optionally, the extension member can be adapted to carry spaced apart longitudinal electrode surfaces for delivery of Rf current to each transected tissue margin from the just-transected medial tissue layers to surface layers.

For example, the combination of the translatable extension member in cooperation with the paired flexible guide members can provide electrode surface engagement with the tissue margins to accomplish the electrosurgical welding technique of the invention. In one embodiment, certain spaced apart portions of channels in the extension member carry electrode surfaces coupled to an Rf source. Thus, when the extension member is moved to the extended position after transecting the engaged tissue volume, one elongate electrode carried at the center of the extension member will engage the medial or interior layers of the transected margin. Other outboard portions of the extension member carry electrodes that engage opposing surfaces of the engaged tissue. By this means, bi-polar current flows can be directed from the center portion of the extension member that engages medial or sub-fascial tissue layers to outward portions of the extension member (or the guides) that engage opposing surface or fascial tissue layers of the targeted tissue volume. It has been found that by engaging the medial portion of a just-transected structure with a first polarity electrode, and engaging the exterior surfaces of the structure with second polarity electrodes, a substantially uniform current flow through non-uniform fascia layers can be accomplished. This novel medial-to-surface bi-polar approach of the invention also reduce or prevent tissue charring, and substantially prevents collateral thermal damage in the tissue by reducing stray Rf current flow through tissue lateral to the engaged tissue.

In another embodiment of the invention, the working end includes components of a sensor system which together with a power controller can control Rf energy delivery during a tissue welding procedure. For example, feedback circuitry for measuring temperatures at one or more temperature sensors in the working end may be provided. Another type of feedback circuitry may be provided for measuring the impedance of tissue engaged between various active electrodes carried by the working end. The power controller may continuously modulate and control Rf delivery in order to achieve (or maintain) a particular parameter such as a particular temperature in tissue, an average of temperatures measured among multiple sensors, a temperature profile (change in energy delivery over time), or a particular impedance level or range.

In another embodiment of the invention, the working end carries one or more indicator mechanisms for indicating to the surgeon when the extension member has been fully extended to close the jaws along their entire length. The indicator mechanism can provide an audio signal, a tactile signal or a light signal. An alternative indicator mechanism is a type of spring-type pop-up indicator.

Additional objects and advantages of the invention will be apparent from the following description, the accompanying drawings and the appended claims.

### BRIEF DESCRIPTION OF THE DRAWINGS

FIG. 1A is an illustration of Rf current flow between the paired jaws of a prior art bi-polar radiofrequency device in a method of sealing a tissue with fascia layers that are resistant to current flow therethrough.

FIG. 1B illustrates representative weld effects of the bi-polar current flow of FIG. 1A.

FIG. 2A is a perspective view of a Type “A” working end of the present invention showing first and second guide members extending from the distal end of an introducer, with a cooperating slidable extension member in a retracted position within the introducer.

FIG. 2B is perspective view of the distal end of the slidable extension member of FIG. 2A with the lower guide member in phantom view, also showing the distal cutting electrode.

FIG. 2C is another view of the working end of FIG. 2A with the extension member moved toward an extended position over guide members.

FIG. 3 is sectional view of a guide member of the invention showing exemplary tissue-gripping elements.

FIGS. 4A-4C are illustrations of initial steps of practicing the method of the invention; FIGS. 4A-4B depicting the positioning of the guide members over a targeted transection path in an anatomic structure, and FIG. 4C depicting the advancement of the extension member over the guide tracks.

FIG. 5 is an enlarged cross-sectional view of the extension member of FIG. 2B showing the electrode arrangement carried by the extension member.

FIG. 6 is a sectional illustration of the extension member of FIG. 5 illustrating the manner of delivering bi-polar Rf current flow to seal or weld a transected tissue margin under high compression.

FIG. 7 is a perspective view of an alterative embodiment of working end that carries an indicator mechanism.

FIG. 8 is a perspective view of another embodiment of working end that carries another indicator mechanism.

FIG. 9 is a view of another embodiment of working end that carries a mechanical indicator.

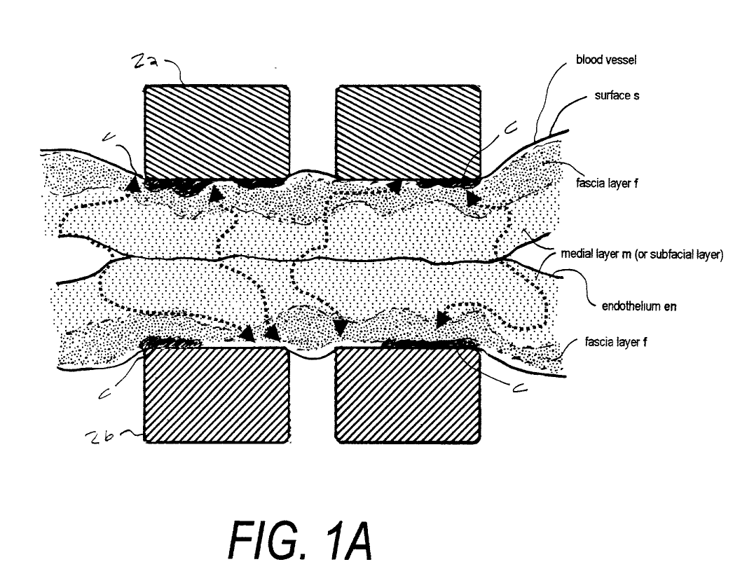

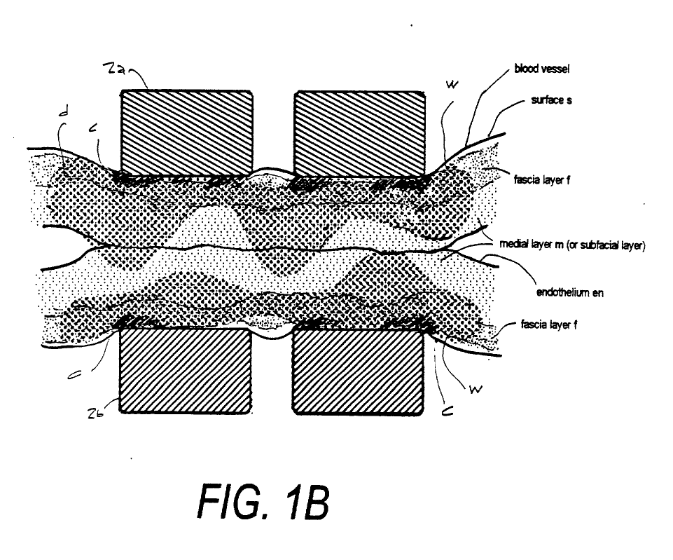

### DETAILED DESCRIPTION OF THE INVENTION

1. Type “A” Working End for Transecting Tissue and Sealing the Transected Margins.

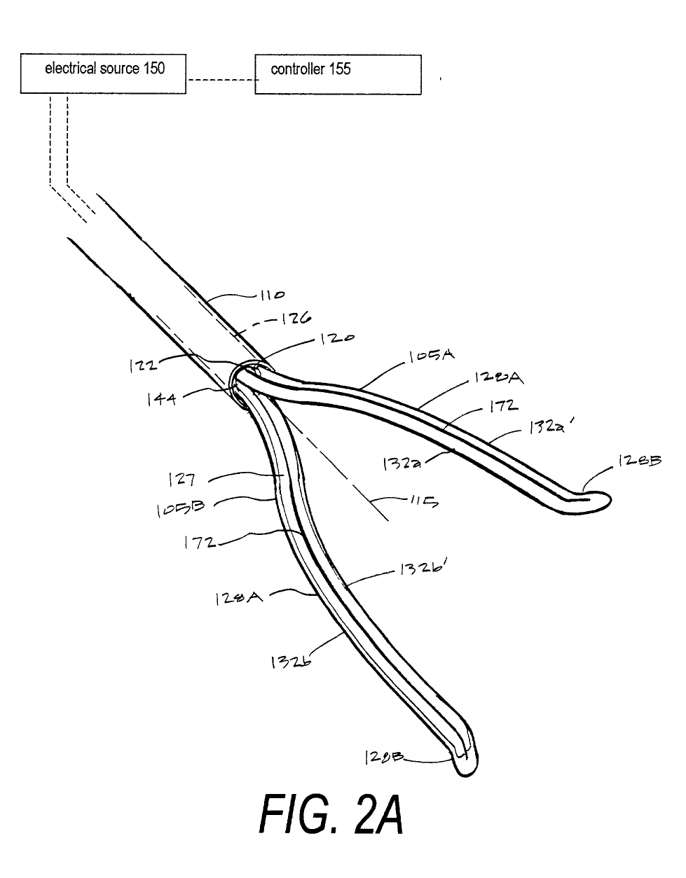

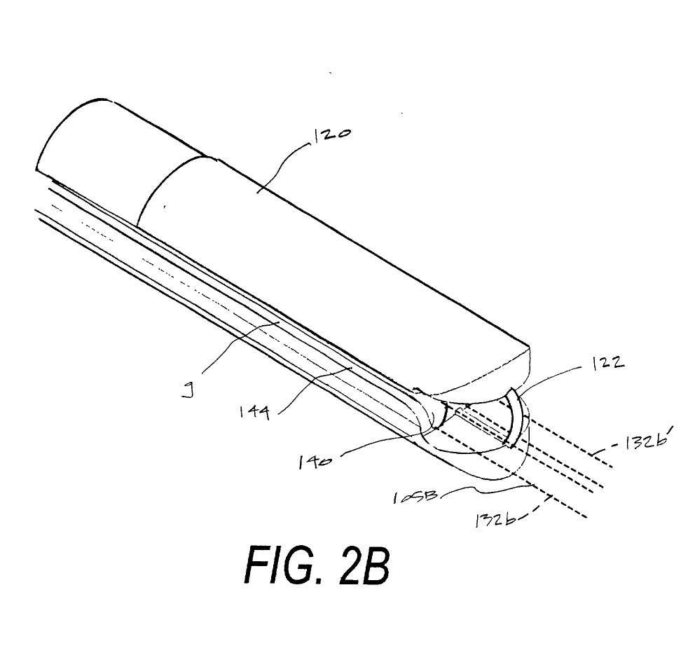

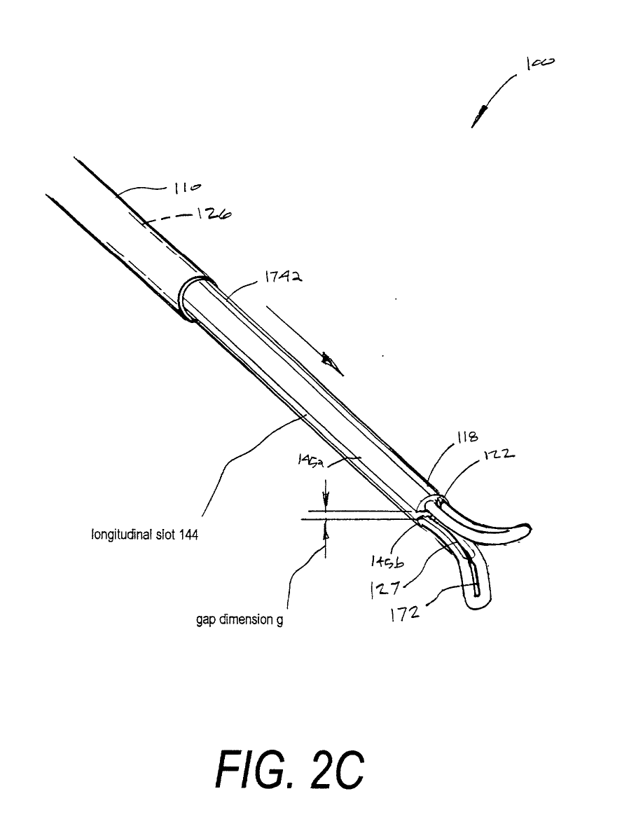

Referring to FIG. 2A, the working end 100 of an exemplary Type “A” embodiment is shown that is adapted for transecting and welding at least one transected tissue margin along a targeted track or path p in tissue, such as lung portion, in an open or endoscopic procedure. The working end 100 has first and second elongate guide members indicated at 105A and 105B that are substantially flexible wire-type elements carried at distal end 108 of an introducer member 110 extending from a proximal handle (not shown). In this Type “A” embodiment, the guide members (or jaws) 105A and 105B extend along a central longitudinal axis 115 and provide multiple functionality: (i) to place over or about a target path p in tissue that is to be transected; (ii) to thereafter guide the terminal portion 118 of an extension member 120 carrying an electrode cutting element 122 along the targeted path p in tissue, and (iii) to provide engagement surfaces 127 for the high-compression engagement of the margins of the transected tissue on both left and right sides of the working end in combination with extension member 120.

In the exemplary embodiment of FIG. 2A, the structural component of introducer portion 110 has a cylindrical cross-section and comprises a thin-wall tubular sleeve (with bore 126) that extends from the proximal handle, although any cross section may be suitable. The diameter of introducer sleeve 110 may range from about 3 mm. to 6 mm., although larger diameter sleeves fall within the scope of the invention. The handle may be any type of pistol-grip or other type of handle known in the art that carries actuator levers or slides to translate the extension member 120 within bore 126 and over the guide members 105A and 105B.

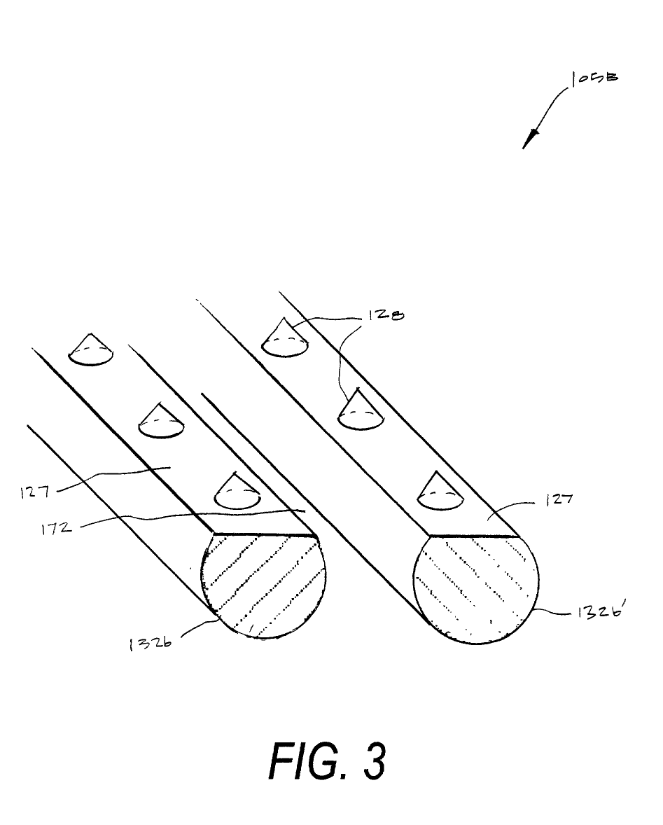

As can be seen in FIG. 2A, one embodiment of the working end 100 has very elongate guide members 105A and 105B of a flexible round wire or rod element, for example, having a diameter ranging from about 0.03″ to 0.10″. The cross-section of guide members 105A and 105B can provide engagement surfaces 127 (collectively) that are flat as shown in FIGS. 2A & 3. Additionally, the surface 127 can carry and type of serrations, sharp projecting elements or any suitable gripping surface better engage tissue as the extension member 120 is advanced over the guides 105A and 105B. FIG. 3 shows exemplary projecting elements 128 (i.e, spikes) that can be provided in the engagement surfaces 127.

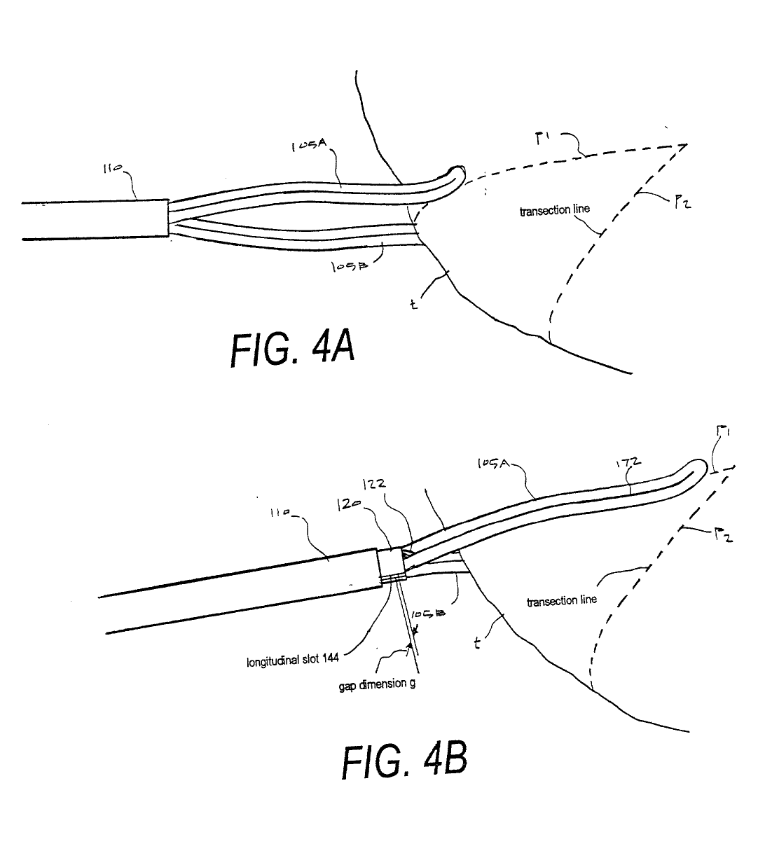

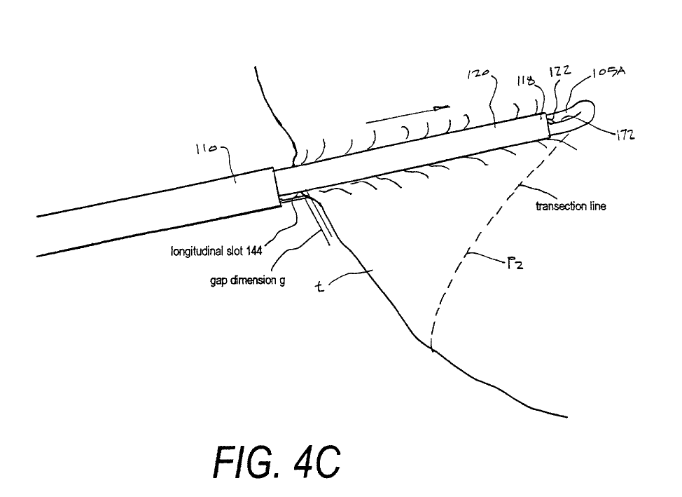

The guide members 105A and 105B in this embodiment define medial outward bowed portions or curve portions indicated at 128A and optional distal angled portions 128B that are adapted to allow guide members 105A and 105B to be pushed over a path p in tissue (see FIG. 4B). It should be appreciated that the shape of the guide members 105A and 105B may be any suitable linear or curved shape to allow ease of placement over a tissue volume targeted for transection. FIGS. 4A-4C illustrate the initial steps of the method of advancing the elongate guide members 105A and 105B over a targeted path in an anatomic structure. FIG. 4A indicates that successive transections along paths p₁ and p₂ can thus accomplish a wedge resection of a targeted tissue volume while at the same time selectively sealing one or both of the transection margins on either side of each path p.

FIGS. 2A & 2C illustrate that guide members 105A and 105B preferably are fabricated of a spring-type metal rod formed with suitable curves 128A and 128B. The guide members 105A and 105B do not comprise jaws in the conventional sense since they are substantially flexible and hence lack jaw-type functionality. That is, the guide members 105A and 105B cannot be moved to a closed position to capture tissue as they provide no inherent strength to be moved between such open and closed positions. Rather, the rod-type elements that make up guide members 105A and 105B are adapted only to guide extension member 120 and to serve as a ramp over the tissue to allow the advancement of extension member 120 over the tissue that otherwise would not be possible.

Referring to FIG. 2B, the extension member 120 slides over the rod-type guide elements with its terminal cutting element 122 transecting the tissue, in which process the extension member 120 captures the combination of the transected tissue margins and the guide members 105A and 105B in a high compression sandwich-like arrangement. It has been found that this means of engaging tissue margins is ideally suited for welding tissue with Rf current. In the exemplary embodiment, the rod-like elements of guide members 105A and 105B comprise paired wire elements, for example, indicated as elements or rods 132a and 132a′ in guide member 105A and rods 132b and 132b′ in guide member 105B (see FIG. 2A). While a metal is a preferred material for guide members 105A and 105B, plastic or composite materials also can be used.

All of the electrosurgical cutting and sealing functionality of the invention is provided in extension member 120 and is described next. As can be seen in FIGS. 2B, 4B-4C & 5, the extension member 120 has a round exterior cross-section and has a first retracted position within the introducer sleeve 110 (see FIG. 2A). FIGS. 2B & 4C show views of the extension member 120 in an extended position as it is being advanced toward a second fully extended position over the guide members 105A and 105B. It can be understood how delivery of high voltage current from an electrical source 150 to the distal cutting element 122 in the terminal portion 118 of the extension member 120 transects the captured tissue t as the member is advanced.

Now turning to FIGS. 2B, 2C & 5, the sectional views of extension member 120 show how the various functional components cooperate. In the embodiment depicted in FIGS. 2B & 5, it can be seen that the extension member 120 has left and right channel portions indicated at 140 (collectively) that are shaped to closely fit around the round rod-type elements of guide members 105A and 105B as the member 120 is slidably moved from its first retracted position toward the second fully extended position.

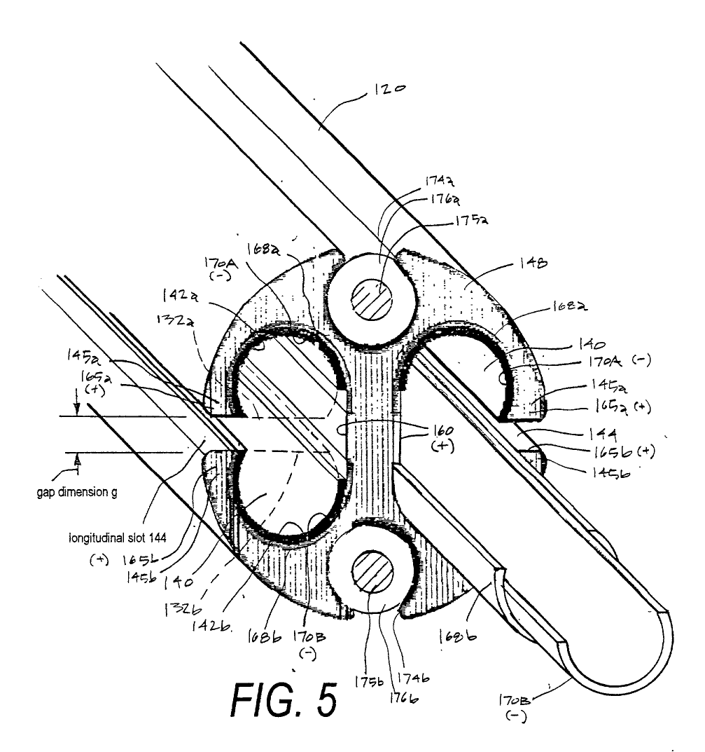

For example, FIG. 5 shows channel 140 at the right side of the instrument (left in view) that has upper surface portions 142a about its top and side that slidably engage one element (132a) of guide member 105A about exterior surfaces of that round element. Likewise, FIG. 5 shows a lower part of the channel 140 with surface portions 142b about the bottom and side of another element (132b) of the lower guide member 105B that slidably engages an exterior of that element. It thus can be seen how the extension member slides over guide members 105A and 105B and flexes the guide members toward one another to allow the entire assembly to compress very tightly about the opposing surfaces of the captured tissue t as the leading edge electrode 122 transects the tissue. The extension member 120 defines a longitudinal slot 144 that extends from each channel 140 to an exterior of the extension member that receives the tissue margins. The slot 144 of extension member 120 thus defines a predetermined gap dimension indicated at g that comprises a selected dimension, and along with the guide members, determines the extent to which the captured tissue will be compressed (see FIGS. 4C & 5). The distal end of the gap g (see FIG. 2B) preferably tapers from a more open dimension to a tighter dimension to initially allow the extension member to slide over engaged tissue. The extension member 120 further defines laterally outward portions 145a and 145b above and below slot 144 that engage the tissue margin. It has been found that tissue should be compressed under high forces for effective Rf welding and the gap g can be substantially small for many tissues. It can be appreciated that the extension member in combination with guide members 105A and 105B can apply very high compressive forces over a long path in tissue for purposes of transection that would not possible with a conventional jaw-type instrument.

The extension member 120 depicted in FIG. 5 can be fabricated by in alternative materials (either plastic or metal) by extrusion processes known in the art, or it can be made by various casting methods if made in a conductive metal. One preferred embodiment as depicted in FIG. 5 provides a body 148 of the extension member that is fabricated of any suitable conductive material such as a metal. The proximal end of the extension member 120 is coupled by an electrical lead (not shown) to an electrical source 150 and controller 155. Thus, the extension member 120 carries electrical potential to serve as an electrode body. The body 148 of the extension member has cooperating electrode surface portions 160 and 165a-165b that are exposed to contact the captured tissue: (i) at the transected medial tissue that interfaces the exposed electrode surface indicated at 160, and (ii) at opposed exterior surfaces of the captured tissue that interface the exposed electrode surfaces 165a and 165b at upper and lower portions (145a and 145b) of extension member 120 outboard (laterally outward) of channels 140. For purposes of illustration, these exposed electrode surface portions 160 and 165a-165b are indicated in FIG. 5 to have a positive polarity (+) to cooperate with negative polarity (−) electrodes described next. These opposing polarity electrodes are, of course, spaced apart from one another and coupled to the electrical source 150 that defines the positive and negative polarities during operation of the instrument. In FIG. 5, it should be appreciated that the left and right sides of the extension member are mirror images of one another with reference to their electrode arrangements. Thus, sealing a tissue margin on either side of the extension member is independent of the other-after the targeted tissue is transected and captured for such Rf welding or sealing as in FIG. 4C. For simplicity, this disclosure describes in detail the electrosurgical methods of sealing a transected tissue margin on one side of the extension member, with the understanding that mirror image events also (optionally) occur on the other side of the assembly.

Still referring to FIG. 5, thin insulator layers 168a and 168b of any suitable plastic or ceramic extend in a partial radius around upper and lower portions of channel 140. Inward of the thin insulator layers 168 are opposing (−) polarity electrodes 170A and 170B that constitute radial sections of elongate hypotubes fitted in the channel and therefore comprise inner surface portions of the channel 140. These longitudinal negative (−) polarity electrodes 170A and 170B, for example of stainless steel, provide the additional advantage of being durable for sliding over the rod elements 132a and 132b that make up portions of guides 105A and 105B. It can be seen that all electrical connections are made to extension member 120 which carries the actual opposing polarity electrodes, thus simplifying fabrication and assembly of the component parts of the working end.

As described above, the distal terminal portion 118 of extension member 120 carries an electrode cutting element indicated at 122 in FIGS. 2B, 4B & 4C. In FIG. 2B, it can be seen that electrode cutting element 122 moves with the longitudinal space 172 between the paired rod-type elements that comprise each guide member 105A and 105B.

FIG. 5 shows that grooves 174a and 174b are provided in the extension member 120 to carry electrical leads 175a and 175b to the cutting electrode 122. These electrical leads 175a and 175b are insulated from the body 148 of extension member 120 by insulative coatings indicated at 176a and 176b.

Now turning to FIGS. 4C & 6, the operation and use of the working end 100 of FIG. 2A in performing a method of the invention can be briefly described as follows. FIG. 4C depicts the extension 120 being advanced from a proximal position toward an extended distal position as it ramps over the tissue by advancing over the guide-track members that compress the tissue just ahead of the advancing extension member. The laterally-outward portions 145a and 145b of the extension member thereby slide over and engage the just-transected tissue margins contemporaneous with the cutting element 122 transecting the tissue. By this means, the transected tissue margins are captured under high compression by working end components on either side of the margins. FIG. 5 thus depicts the targeted tissue margins t captured between upper and lower portions of the extension member outward of channels 140. The targeted tissue t may be any soft tissue or anatomic structure of a patient's body. The targeted tissue is shown with a surface or fascia layer indicated at f and medial tissue layers m. While FIGS. 4B-4C depict the tissue being transected by a high voltage Rf cutting element 122, it should be appreciated that the cutting element also can be a blade member.

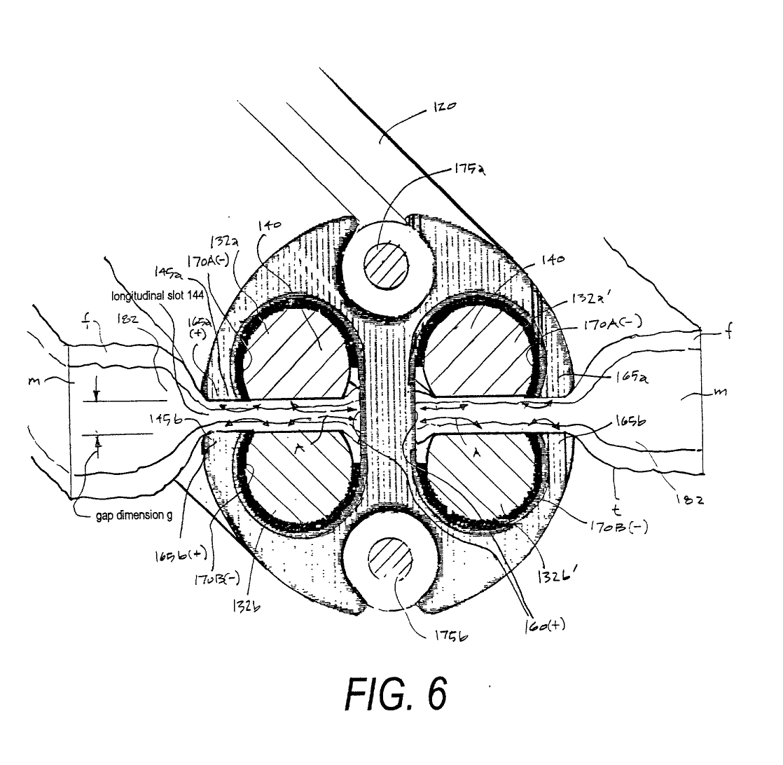

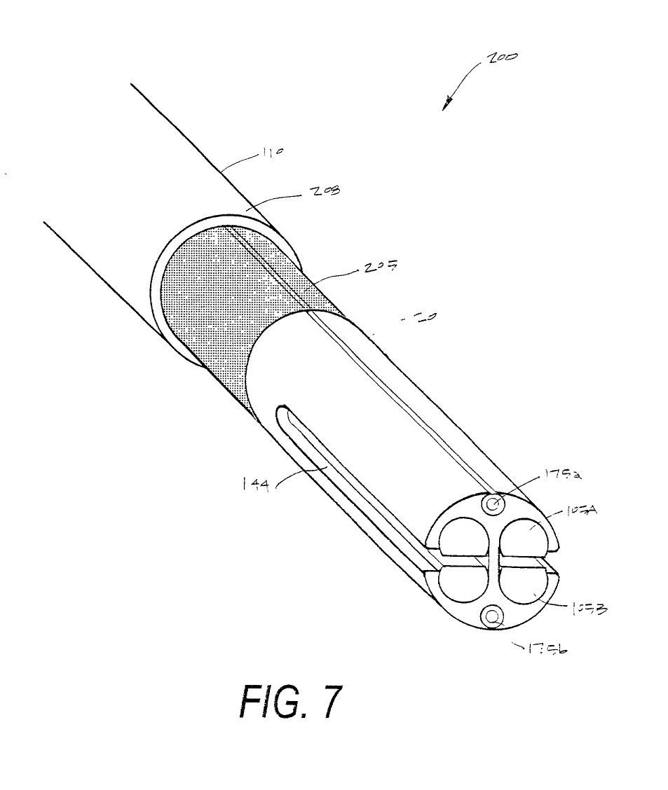

FIG. 6 provides an illustration of one preferred manner of Rf current flow that causes a sealing or welding effect by the medial-to-surface bi-polar current flow (or vice versa) indicated by arrows A. It has been found that a substantially uniform weld can be created across the captured tissue margin by causing current flow from exposed electrode surfaces 165A and 165B to the electrodes 170A and 170B that further conducts current flow through conductive guide rod elements 132a and 132b. In other words, the sectional illustration of FIG. 6 shows that a weld can be created in the captured tissue margin where proteins (including collagen) are denatured, intermixed under high compressive forces, and fused upon cooling to seal or weld the transected tissue margin. Further, it is believed that the desired weld effects can be accomplished substantially without collateral thermal damage to adjacent tissues indicated at 182 in FIG. 6.

Another embodiment of the invention (not shown) includes a sensor array of individual sensors (or a single sensor) carried in any part of the extension member 120 or guide member 105A-105B that contacts engaged tissue. Such sensors preferably are located either under an electrode 170A-170B or adjacent to an electrode for the purpose of measuring temperatures of the electrode or tissue adjacent to an electrode during a welding procedure. The sensor array typically will consist of thermocouples or thermistors (temperature sensors that have resistances that vary with the temperature level). Thermocouples typically consist of paired dissimilar metals such as copper and constantan which form a T-type thermocouple as is known in the art. Such a sensor system can be linked to feedback circuitry that together with a power controller can control Rf energy delivery during a tissue welding procedure. The feedback circuitry can measure temperatures at one or more sensor locations, or sensors can measure the impedance of tissue, or voltage across the tissue, that is engaged between the electrodes carried by the working end. The power controller then can modulate Rf delivery in order to achieve (or maintain) a particular parameter such as a particular temperature in tissue, an average of temperatures measured among multiple sensors, a temperature profile (change in energy delivery over time), a particular impedance level or range, or a voltage level as is known in the art.

2. Type “B” Working End.

Another embodiment of the invention includes an indicator system that provides the surgeon with one or more signals that the guide members or jaws have been moved to the second fully closed position or another selected intermediate position. It has been found that when the working end of the invention is configured with very elongate jaws and used in an endoscopic surgery, it can be difficult for the surgeon to determine when the extension member is moved to the second fully extended position to close the distalmost portions of the jaws. In such an endoscopic surgery, it may not be possible to manipulate the endoscope to view the distal portion of elongate jaws at the same time as viewing more proximal portions of the working end and the targeted tissue. Since the method of the invention utilizes very high compressive forces applied to engaged tissues together with Rf delivery to create an effective weld, the operator must know when the distalmost jaw portions engage tissue under high compression. For this reason, the invention provides at least one indicator system to inform the surgeon when the extension member is fully extended to the second extended position, or alternatively to another selected position.

One indicator system comprises an audio signal emitter of a type known in the art that can be carried in the handle of the instrument. The extension member 120 of the invention (FIG. 5) reciprocates in a housing in a handle and within bore 126 of the introducer sleeve 110. The extension member 120 and sleeve 110 can carry electrical contacts (not shown) that are coupled when the extension member 120 is advanced to the second extended position or another selected position. The audio tone emitter then can be activated by the closing of the electrical contacts. Alternatively, the audio signal can be a spring-type element carried by the extension member or instrument body that emits an audible “click” when the extension member is extended to a selected position and the spring-type element snaps into, or out of, a notch in the cooperating components. The instrument also can be provided with a light emitter that indicates when the extension member is extended to a selected position. For example, a light emitting diode (LED) can be carried at the distal end of the introducer sleeve. Alternatively, one or more LED's can be provided at locations in the handle, the proximal portion of the introducer sleeve or the distal end of the extension member. As another alternative, the instrument can be provided with a tactile emitter that vibrates the handle slightly when the extension member is extended to the selected position.

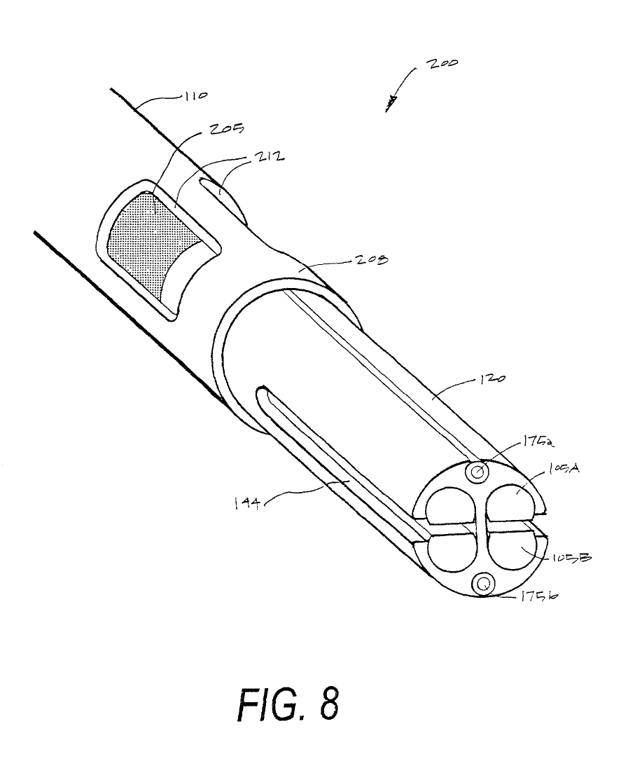

FIG. 7 depicts another embodiment of working end 200 that carries an indicator comprising a visual marking 205 that is exposed to view when the extension member is fully extended to the second position. In FIG. 7, the extension member 120 has a color marking that is exposed beyond the distal end 208 of introducer sleeve 110 that can function as an indicator mechanism. It should be appreciated that the visual marking 205 can be any suitable type of surface marking such as a color, a scribed marking, a surface reflectivity difference as a marking, a texture difference as a marking, or any other visual marking. FIG. 8 depicts an alternative working end that carries at least one aperture or window 212 in the distal portion of the introducer sleeve 110 that exposed a color marking 205 to the view of the surgeon. It should be appreciated that such an aperture 212 and color marking on the extension member also can be provided in a proximal portion of the instrument.

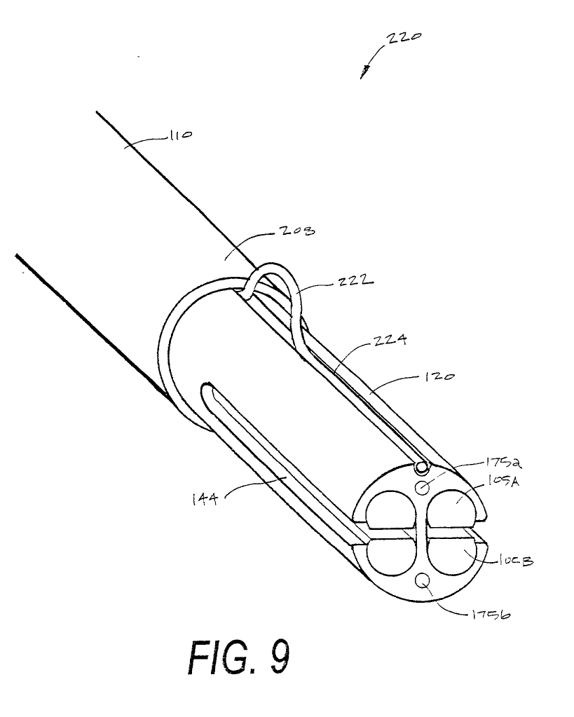

FIG. 9 depicts another preferred embodiment of working end 220 that carries a “pop-up” type mechanical indicator at the distal end 208 of the introducer sleeve 110. In the embodiment of FIG. 9, the upper portion of the extension member 120 can carry a leaf-type spring member 222 in a receiving channel portion 224 of the member. When the extension member 120 is in a retracted position within the introducer sleeve 110, the spring member is in a tensioned state. When the extension member 120 is extended beyond the distal end 208 of the introducer sleeve 110, the spring member 222 that can be released to its repose position of FIG. 9 which will be visible to the operator.

The above sections have described various signal mechanisms that are adapted to inform the surgeon that the extension member has been moved to an extended position so that the extension member and guides are engaging the targeted tissue under high compression. Thereafter, the surgeon actuates a switch to deliver Rf current to the working end to weld the engaged tissue. Another embodiment of the invention provides an interlock or interconnect between any selected type of sensing mechanism that thereafter generates a signal, and the controller 155 and electrical source 150 for enabling Rf current delivery to the tissue-welding electrode arrangement. For example, as described above, the slidable extension member 120 can have an electrical contact that couples with another contact in a housing to sense when the extension member is advanced to a selected position. In one embodiment, the controller 155 and circuitry that delivers Rf current to the tissue-welding electrodes will be disabled until the extension member is advanced to a selected position. Thus, the sensing mechanism can be linked with an interlock mechanism that enables Rf energy delivery only after the extension member is in the selected position. This interlock system will prevent the surgeon from delivering Rf current to the tissue-welding electrodes before the jaws are fully closed. Another type of interconnect system can automatically deliver Rf current to the tissue-welding electrodes when the extension member is advanced to the fully extended position. The instrument further can be provided with a selector switch to allow the surgeon to choose between manual or automatic delivery of Rf current to the tissue-welding electrodes when the jaws are fully closed.

Another embodiment of the system provides a circuitry interconnect system that delivers Rf energy to the distal cutting electrode 122 automatically as long as the extension member 120 is being advanced. The system also would terminate energy delivery to the cutting electrode 122 when the extension member reached a fully extended position.

Although particular embodiments of the present invention have been described above in detail, it will be understood that this description is merely for purposes of illustration. Specific features of the invention are shown in some drawings and not in others, and this is for convenience only and any feature may be combined with another in accordance with the invention. Further variations will be apparent to one skilled in the art in light of this disclosure and are intended to fall within the scope of the appended claims.

What is claimed is:

1. An electrosurgical transecting-sealing instrument, comprising:
a handle member coupled to an introducer member with paired elongate guide members extending along an axis from the distal end of the introducer member;
a slidable extension member that is axially moveable from a first retracted position to a second extended position relative to the paired guide members to move said guide members from a first open position to a second closed position, the extension member defining axial channel surfaces that engage outer surfaces of said guide members;
a cutting element carried at the distal termination of the extension member;
opposing polarity electrodes carried in the guide members and extension member; and
an indicator mechanism in the instrument that indicates when the paired guide members are moved to a selected position.

2. The instrument of claim 1 wherein said selected position is the second closed position or a position intermediate the first open position and the second closed position.

3. The instrument of claim 1 wherein the indicator mechanism comprises an audio signal emitter.

4. The instrument of claim 1 wherein the indicator mechanism comprises a light emitter.

5. The instrument of claim 4 wherein the light emitter is in a location selected from locations in the handle, the proximal end of the introducer member, the distal end of the introducer member and the distal portion of the extension member.

6. The instrument of claim 1 wherein the indicator mechanism comprises a tactile emitter mechanism in the handle member.

7. The instrument of claim 1 wherein the indicator mechanism comprises a visual indicator on a portion of said extension member that extends distally from the introducer member when said extension member is moved to said selected position.

8. The instrument of claim 6 wherein the visual indicator is selected from the class of color markings, scribed markings, reflectivity markings, or surface texture markings.

9. The instrument of claim 1 wherein the indicator mechanism comprises a visual indicator on a portion of said extension member that is exposed through an aperture in a body portion of the instrument when the extension member is fully moved to said second extended position.

10. The instrument of claim 9 wherein said aperture is in a location selected from locations in the handle, the proximal end of the introducer member and the distal end of the introducer member.

11. The instrument of claim 9 wherein the visual indicator is selected from the class of color markings, scribed markings, reflectivity markings, or surface texture markings.

12. The instrument of claim 1 wherein the indicator mechanism comprises a spring-type indicator carried within said extension member that is in a tensioned position when the extension member is in a first retracted position and an untensioned position when the extension member is fully extended to the second extended position.

13. The instrument of claim 12 wherein said spring-type indicator extends outwardly from the instrument body when the extension member is fully extended to the second extended position.

14. An electrosurgical instrument, comprising:
a handle portion coupled to a housing body extending to a working end that carries first and second jaws;
a slidable extension member moveable from a first retracted position to a second extended position in a bore in said housing body to move the first and second jaws from an open position to a closed position;
an electrode cutting element carried at the distal termination of the extension member;
opposing polarity electrodes carried in the working end; and
a sensing mechanism that senses when the jaws are moved to said second closed position.

16. The instrument of claim 15 wherein the sensing mechanism comprises electrical contacts carried by the extension member and the housing body.

17. The instrument of claim 15 wherein the sensing mechanism is coupled to indicator mechanism selected from the class consisting of audio signal generating mechanisms, tactile signal generating mechanisms, and light emitter mechanisms.

18. The instrument of claim 15 wherein the sensing mechanism is coupled to interlock circuitry to enable delivery of electrical energy to said opposing polarity electrodes only when said extension member is moved to a selected extended position.

19. The instrument of claim 15 wherein the sensing mechanism is coupled to interconnect circuitry to automatically deliver electrical energy to said opposing polarity electrodes when said extension member is moved to a selected extended position.

20. An electrosurgical instrument, comprising:
a handle portion coupled to a housing body extending to a working end that carries first and second jaws;
a slidable extension member moveable from a first retracted position to a second extended position in a bore in said housing body to move the first and second jaws from an open position to a closed position;
an electrode cutting element carried at the distal termination of the extension member;
opposing polarity electrodes carried in the working end; and
a sensor mechanism in said extension member and housing body that senses the jaws are moved to said second closed position to enable circuitry to deliver electrical energy to said opposing polarity electrodes.
